(19) United States
(12) Patent Application Publication
(10) Pub. No.: US 2024/0298609 A1
(43) Pub. Date: Sep. 12, 2024

(54) K9 QUICK-CLIP OXYGEN MUZZLE

(71) Applicant: Thomas Kenney Barrett, JR., Midlothian, VA (US)

(72) Inventor: Thomas Kenney Barrett, JR., Midlothian, VA (US)

(21) Appl. No.: 18/656,716

(22) Filed: May 7, 2024

Related U.S. Application Data
(63) Continuation-in-part of application No. 17/550,752, filed on Dec. 14, 2021.

(51) Int. Cl. A01K 25/00; A61M 16/06

(52) U.S. Cl. CPC A01K 25/00; A61M 16/0683; A61M 2202/0208; A61M 2250/00

(57) ABSTRACT

An oxygen muzzle is capable of protecting the canine handler from a bite while providing lifesaving oxygen to a canine in distress. The muzzle is designed to adjustably fasten behind the head of a canine while delivering needed oxygen through a dedicated input port and tubing designed for quick hook up.

### REFERENCE TO RELATED APPLICATIONS

This is a continuation-in-part application claiming the priority of non-provisional application Ser. No. 17/550,752 filed Dec. 14, 2021 titled K9 Quick Clip Oxygen Muzzle, which claims the priority of non-provisional application Ser. No. 16/292,558 filed Jul. 25, 2019 titled K9 Quick Clip Oxygen Muzzle which claims the priority of provisional application No. 62/761,047 filed on Mar. 5, 2018 titled K9 Quick Clip Oxygen Muzzle.

### BACKGROUND OF THE INVENTION

This invention relates generally to oxygen therapy for canines and, more particularly, to an oxygen muzzle configured to quickly attach/clip to a dog's snout. In other words, this invention supplies rapid oxygen therapy to a service dog.

As canines have become more involved in military roles, law enforcement, and search and rescue roles, a greater emphasis on medical procedures has been experienced. Medical protocols have been implemented to deal with more frequent emergencies. In the same manner humans do, canines require sufficient and constant supplies of oxygen to survive. Dogs, like humans may experience respiratory distress which may be made worse if the canine is experiencing challenges such as pneumonia, congestive heart failure, or rigorous exertion in dry or unsanitary environments. When serving in the military or law enforcement, canines may find themselves in remote areas where their need of rapid oxygen therapy is simply not available. In such cases, providing additional oxygen can be a lifesaving intervention, aiding in the recovery process and improving overall quality of life.

There is one known item used for oxygen therapy for canines in an urban environment but not in a working field environment. It is a hard plastic cone with an oxygen hook-up. However, it is neither compactable nor flexible.

Therefore, it would be desirable to have a dual-purpose muzzle capable of protecting the canine handler from a bite while providing lifesaving oxygen to a canine in distress. Further, it would be desirable to have a dual-purpose muzzle having a quick connect construction for positioning on the nose or snout of a dog. In addition, it would be desirable to have a dual-purpose muzzle that is both compatible and flexible.

### SUMMARY OF THE INVENTION

Accordingly, a quick action oxygen muzzle for providing oxygen therapy to a canine according to the present invention includes a muzzle having a nose portion adjacent and connected to an adjustment portion, the muzzle having a conical configuration defining an open end for receiving a snout of the canine.

The nose portion being constructed of an opaque, non-permeable material that is flexible and the adjustment portion being constructed of a transparent plastic membrane so that oxygen flow can be verified. The nose cone may be flexible and the adjustment portion defining the open end. The oxygen muzzle includes a length of tubing having an elongated configuration for gaseous transmission, the tubing having a proximal end proximate an interior area defined by the nose portion and a distal end displaced and extended away from the proximal end. The proximal end of the tubing includes at least one outlet port through which oxygen is selectively expelled. Further, a pouch is coupled to the distal end of the tubing and the receptacle defines an interior space. An oxygen container is situated in the interior space of the receptacle and in fluid communication with the tubing so as to push oxygen toward the at least one outlet port when actuated. A harness system may also be included for holding the muzzle to the canine and for selectively attaching the receptacle itself to the canine.

Therefore, a general object of this invention is to provide an oxygen muzzle that may be rapidly and easily administered to a canine.

Another object of this invention is to provide an oxygen muzzle, as aforesaid, that clips behind the head of the canine like a soft muzzle having external tubing and a port for fast hook up to an oxygen source, such as may be provided by an Emergency Medical Service (EMS) provider.

A further object of this invention is to provide an oxygen muzzle, as aforesaid, that includes transparent windows such that oxygen flow can be visually verified as well as to make the oxygen muzzle lightweight and tactical.

A still further object of this invention is to provide an oxygen muzzle, as aforesaid, that is portable, lightweight, and may be folded and stowed.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1a is a perspective view a muzzle of an oxygen muzzle according to a preferred embodiment of the present invention, said snout portion being shown with an adjustment portion in a fully extended configuration; and

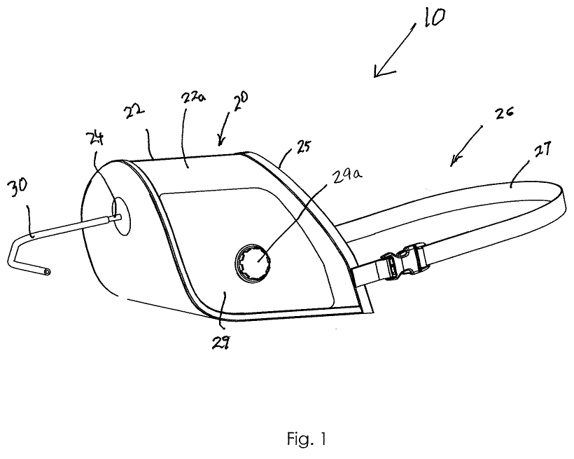

FIG. 1b is a perspective view of the muzzle as in FIG. 1a, illustrated with an adjustment portion in a partially compressed configuration;

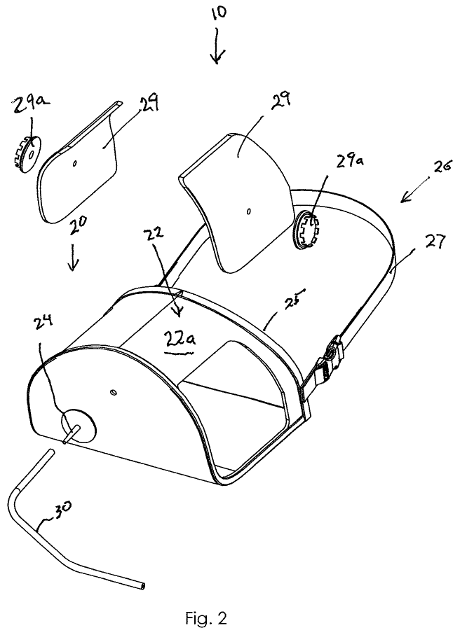

### DESCRIPTION OF THE PREFERRED EMBODIMENT

A K9 quick clip oxygen muzzle according to a preferred embodiment of the present invention will now be described with reference to the accompanying drawings.

The oxygen muzzle 10 includes a construction that makes it conducive to be collapsed and folded for stowage when not in use, such as would enable the muzzle 10 to be carried in a person's pocket so as to be available if a user accompanies a canine on a military or law enforcement mission that takes them far away from veterinary care. More particularly, the oxygen muzzle 10 includes a muzzle 20 having a nose portion 22 connected to an adjustment portion 26. More particularly, the nose portion 22 has a rounded front end 23 and an open rear edge (also referred to as an open end 25 or an open rear end), the adjustment portion 26 including a head strap 27 coupled to the nose portion 22 so as to span the open end 25 such as by stitching. Together, the nose portion 22 of the muzzle 20 has a conical configuration defining an interior area, a rear edge of the adjustment portion 26 defining an opening sufficient for receiving a snout (nose) of a canine into the interior area.

Described in even greater detail, the nose portion 22 includes a body portion that extends rearwardly from the front end 23 and that defines the conical configuration and the interior area inside. In an embodiment, the nose portion 22 may be constructed of flexible plastic materials so as to be movable between a collapsed configuration or partially collapsed configuration more adjacent or more proximate the nose portion 22 and an extended or deployed configuration extending rearwardly and away from the nose portion 22. Accordingly, the nose portion 22 may be folded for storage or transport and then deployed or extended when needed for oxygen therapy.

In a critical aspect, the nose portion 22 may have a construction that enables the canine handler or medical personnel to determine that oxygen is in fact flowing through the inlet port 24 and into the interior area where it may be inhaled by the canine. More particularly, the body portion of the nose portion 22 may include a support section 22a that is constructed of a material that is opaque, non-permeable, and flexible. In an embodiment, the support section 22a may include a top wall, a bottom wall, and the front end 23 of the nose portion 22. This construction provides meaningful shape definition in the open configuration shown in the drawings and that is conducive for receiving the snout of the canine in use.

With further reference to the nose portion 12, the body portion may include at least one and preferably two viewing windows 29 extending between the walls of the support section 22a described above, each viewing window being constructed of a transparent material such as polytetrafluoroethylene (PTFE), polyethylene terephthalate (PET), and bio plastics which are seen as advantageous due to being lightweight, increasingly effective, and inexpensive when compared to glass or the like. Preferably, the pair of viewing windows 29 are laterally spaced apart and opposite one another.

In addition, the nose portion 22 may include a pair of one-way ventilation devices 29a (also referred to as vents) coupled to the viewing windows 29, respectively. Preferably, each vent is configured to expel air from inside the interior area into the ambient air outside of the nose portion 22. Preferably, oxygenated air or even pure oxygen will be inhaled by the canine's snout received in the nose portion 22 during oxygen therapy and will then exhale oxygen-poor air back into the interior area where it will then be expelled through the ventilation devices 29a.

In an aspect, the front end 23 of the nose portion 22 may define or include an inlet port 24 configured for connection to an oxygen source, e.g., an oxygen tank such as may be provided for oxygen therapy. In an embodiment, the inlet port 24 may be in communication with an interior area defined by the nose portion 22. Accordingly, any gas transmitted downstream through the inlet port 24, such as oxygen, will pass into the interior area where the snout of a canine has been inserted. In this way, oxygen is imparted as therapy to a canine.

Described in even more detail, the oxygen muzzle 10 includes a length of tubing 30 having an elongate configuration, said tubing 30 having a diameter so as to allow a gaseous stream, such as oxygen, to flow through the tubing 30. The tubing 30 is configured for removable attachment to the inlet port 24 for directing oxygen from an external tank into the interior area to be inhaled by the canine.

In an embodiment, adjustment portion 26 is configured to be selectively fastened behind a head of the canine or the like. More particularly, the adjustment portion 26 may include a head strap 27 that is constructed of an elastic material that may be stretched behind the head of the canine and which will then self-tighten to secure the snout of the canine in the interior area of the nose portion 22. That it is understood that the elastic material may be in the form of a sturdy textile or semi-rigid like rubber. In another embodiment (not shown), the head strap 27 may include additional straps or buckles configured to adjustably secure the nose portion 22 on the snout of the canine. The head strap 27 may include opposed ends each coupled to the nose portion 22 adjacent opposed edges of the open end 25, such as by stitching.

In use, a canine's handler may desire to administer oxygen therapy to a canine, especially after an intense or lengthy task while still in the field of service and away from traditional veterinary care. In such case the oxygen muzzle 10 according to the present invention may be available for easy transport, fast connection to a canine's snout, and capable of quickly administering oxygen for inhalation by the canine. More particularly, the muzzle 20 may be unfolded, extended, and positioned on the snout of a canine and may be secured in that position by the head strap 27. Oxygen from an external oxygen tank may be received through the tubing 30 and through the input port 24 into the interior area of the nose portion 22 where it may be inhaled by the dog. The transparent windows 29 will enable the handler to confirm that oxygen is, in fact, flowing to the canine. When an oxygen therapy session is complete, the muzzle 10 may be folded and stowed until needed again.

It is understood that while certain forms of this invention have been illustrated and described, it is not limited thereto except insofar as such limitations are included in the following claims and allowable functional equivalents thereof.

1. A quick action oxygen muzzle for providing oxygen therapy to a canine, comprising:
a muzzle comprising a nose portion that includes a front end, a body portion having a conical configuration and that is coupled to and extends rearwardly from said front end, and an open end opposite said front end that is configured for receiving a snout of the canine,
said body portion including (1) a support section constructed of a material that is opaque, non-permeable, and flexible and (2) a pair of viewing windows fixedly coupled to the support section and situated opposite one another;
an input port mounted to said front end of said nose portion that is in fluid communication with an interior area defined by said nose portion;
tubing having a proximal end operatively coupled to said input port and having an elongate configuration for gaseous transmission; and
a pair of vent ports coupled to said pair of viewing windows, respectively, each having a configuration through which oxygen is selectively expelled.

2. The oxygen muzzle as in claim 1, wherein said input port is operable to transmit oxygen from said tubing to said interior area.

3. The oxygen muzzle as in claim 1, further comprising an adjustment portion that includes a head strap having opposed ends coupled to said body portion adjacent opposite edges of said open end, respectively, such that said head strap is operable to attach said muzzle onto the snout of the canine when said at least one strap is coupled about a neck of the canine.

4. A quick action oxygen muzzle for providing oxygen therapy to a canine, comprising:
a muzzle that includes (1) a nose portion having a front end and defining an open end opposite said front end that is configured for receiving a snout of the canine into an interior area defined by said nose portion and (2) an adjustment portion extending across said open end,
said nose portion of said muzzle including a body portion having a conical configuration, said body portion including (1) a support section constructed of a material that is opaque, non-permeable, and flexible and (2) a pair of viewing windows fixedly coupled to the support section and situated opposite one another;
an input port mounted to said front end of said nose portion that is in fluid communication with said interior area;
tubing having a proximal end operatively coupled to said input port and having an elongate configuration for transmitting oxygen; and
a pair of vent ports coupled to said pair of viewing windows, respectively, each having a configuration through which oxygen is selectively expelled;
wherein said adjustment assembly includes a head strap having opposed ends coupled to said body portion adjacent opposite edges of said open end, respectively, such that said head strap is operable to attach said muzzle on the snout of the canine when said head strap is coupled about a neck of the canine.

5. The oxygen muzzle as in claim 4, wherein said front port is operable to transmit oxygen from outside said nose portion into said interior area defined by said nose portion.

6. The oxygen muzzle as in claim 4, wherein said head strap is constructed using an elastic material.
